(19) United States
(12) Patent Application Publication
(10) Pub. No.: US 2025/0277239 A1
(43) Pub. Date: Sep. 4, 2025

(54) A COMPOSITION FOR THE PRODUCTION OF BIOFUEL AND PROCESS OF MAKING SAME

(71) Applicants: Nikolai Holder, Christ Church (BB); Rum and Sargassum Inc, Holders, St. James (BB)

(72) Inventors: Legena Henry, Mesa, AZ (US); Brittney McKenzie, Christ Church (BB); Shamika Spencer, St Pauls Parish (AG); Aria Goodridge, St Peter (BB); Nikolai Holder, Christ Church (BB)

(21) Appl. No.: 18/858,787

(22) PCT Filed: Apr. 24, 2023
(86) PCT No.: PCT/US2023/019574

§ 371 Date: Oct. 22, 2024

Related U.S. Application Data
(60) Provisional application No. 63/333,691, filed on Apr. 22, 2022.

(51) Int. Cl. C12P 5/02; C10J 3/72

(52) U.S. Cl. CPC C12P 5/023; C10J 3/72; C10J 2300/0916

(57) ABSTRACT

A biofuel composition and making of same, wherein the biofuel composition utilizes the co-digestion of multiple substrates to produce energy by combining and converting inexpensive waste streams to useful, inexpensive, sustainable energy by combining and co-digesting a quantity of sargassum seaweed, a quantity of rum distillery wastewater and an inoculum, and wherein the biofuel may be utilized as an alternative transportation fuel.

### CROSS REFERENCE TO RELATED APPLICATION

This application claims the benefit of and takes priority from U.S. Provisional Patent Application Ser. No. 63/333,691 filed on Apr. 22, 2022, the contents of which are herein incorporated by reference.

### BACKGROUND OF THE INVENTION

### Field of the Invention

The present disclosure relates generally to an alternative biofuel derived from biomethane, more particularly from the combination and anaerobic digestion of quantities of Sargassum seaweed, inoculum, and wastewater.

### Description of the Related Art

Efforts are being made globally to cut down the use of fossil fuels and the emission of Carbon in order to reduce the impacts of global warming. There is a growing awareness and emphasis on the carbon-neutral operation of biofuels.

Methane can be obtained by converting organic matter contained in sludge through the anaerobic process of wastewater treatment and other organic agricultural livestock or food wastes. However, the wide application of anaerobic digestion has always been limited due to the low energy conversion efficiency of sludge or organic feedstocks. To overcome this issue and enhance methane production, nanomaterials and marine algae have been recommended as additives to the feedstock after effective trials. Biomass, such as municipal and agricultural waste, still possesses a huge potential to improve industrial anaerobic digestion through the use of nanomaterials, particularly with the use of nanoparticles along with seaweeds, as such macroalgae also capture Carbon and reduce Ocean Acidification while they grow in the marine environment prior to harvest.

Energy consumption has risen incrementally as the continued dedication and growth of society, and the demand for cars and other forms of transportation. A result of increasing energy consumption is the accompanying reliance on fossil fuels to power that consumption.

It is a global objective to reduce the use of fossil fuels and the emission of Carbon into the atmosphere. There is a growing demand for alternative methods of fuel production, especially biofuels. Biofuels are derived from biomass, including plant and animal waste.

Small island developing states (SIDS) have increased demand for fossil fuels to match the increase in energy consumption. This dependency is problematic because fossil fuels must be imported to the SIDS, and energy consumption powered by fossil fuels is linked to climate change form carbon emissions.

Potential alternatives, while effective fuel sources, do not offset carbon emissions completely. For example, electric vehicles and the required charging ports for those vehicles powered by solar energy are costly and financially infeasible for many. Thus, biofuels have been researched as a further alternative.

Biofuels, specifically biomethane, can be produced by the anaerobic digestion of organic wastes or dedicated energy crops. Sugar cane has been used as a biomass in the production of biofuel via a transformation to bioethanol. However, this sort of production of bioethanol involves the use of large amounts of agricultural land, and land availability is scarce for SIDS.

Sargassum seaweed is a naturally occurring seaweed species that is both highly abundant and an invasive irritant to SIDS in the Caribbean Sea. This biomass is abundant and predicted to increase in the coming years. Because it is a seaweed, it requires no agricultural land use and would benefit SIDS who rely on tourism with its removal from beaches.

### SUMMARY OF THE INVENTION

The present composition, as illustrated herein, is clearly not anticipated, rendered obvious, or even present in any of the prior art mechanisms, either alone or in any combination thereof. Thus, the several embodiments of the instant apparatus are illustrated herein.

A primary object of the present disclosure is to produce biomethane for transportation fuel so as to lower the carbon emissions caused by electricity consumption powered by fossil fuels.

Another object of the present disclosure is to utilize the overabundance of the invasive irritant Sargassum seaweed and prevent it from reaching beaches in the Caribbean.

Another object of the present disclosure is to remove sargassum from deep water areas to reduce beach restoration costs and property closures.

Another object of the present disclosure is to repurpose wastewater from rum distilleries and animal waste in the production of biofuel from biomethane.

Another object of the present disclosure is to produce biomethane from a composition of Sargassum seaweed, rum distillery waste, and animal waste though anaerobic digestion.

Another object of the present disclosure is to upgrade biogas from the combination to biomethane and compress the biomethane to refuel compressed natural gas (CNG) vehicles via a standard commercial CNG compressor and dispenser.

Another object of the present disclosure is to produce an inexpensive alternative fuel to provide clean energy.

Yet another object of the present disclosure is to generate biofuel by co-digesting three key waste materials: Sargassum seaweed, rum distillery wastewater and an inoculum.

Yet another object of the present disclosure is to produce energy by combining and converting inexpensive waste streams to useful, inexpensive, sustainable energy.

In this respect, before explaining at least one embodiment of the composition in detail, it is to be understood that the system is not limited in its application to the details of construction and to the arrangements of the components set forth in the following description, and/or illustrated in the drawings. The composition and system is capable of other embodiments and of being practiced and carried out in various ways. Also, it is to be understood that the phraseology and terminology employed herein are for the purpose of description and should not be regarded as limiting.

The foregoing has outlined the more pertinent and important features of the present system in order that the detailed description of the system that follows may be better understood, and the present contributions to the art may be more fully appreciated. It is of course not possible to describe every conceivable combination of components and/or methodologies, but one of ordinary skill in the art may recognize that many further combinations or permutations are possible. Accordingly, the novel architecture described below is intended to embrace all such alterations, modifications, and variations that fall within the spirit and scope of the appended claims.

Variations of the disclosure may be envisioned by those skilled in the art and the invention is to be limited solely by the claims hereto.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram illustrating the collection and treatment of Sargassum seaweed for anaerobic digestion to produce biomethane for use as a biofuel.

FIG. 2 is a schematic diagram illustrating an experimental set-up for a biomethane potential test utilizing a micro-digester.

FIG. 3 is a graph showing the volume of methane (Nml/gFM) produced from sample 2 of Table 1 versus a period of time (days).

FIG. 4 is a graph showing the volume of methane (Nml/gFM) produced from sample 3 of Table 1 versus a period of time (days).

FIG. 5 is a graph showing the volume of methane (Nml/gFM) produced from samples 5 and 12 of Table 1 versus a period of time (days).

FIG. 6 is a graph showing the volume of methane (Nml/gFM) produced from samples 6 and 13 of Table 1 versus a period of time (days).

FIG. 7 is a graph showing the cumulative methane production (Nml/gFM) versus time (days) from all tested samples in Table 1.

FIG. 8 is a schematic diagram illustrating a biomass prediction mobile application utilized to predict the location and arrival of sargassum seaweed for collection.

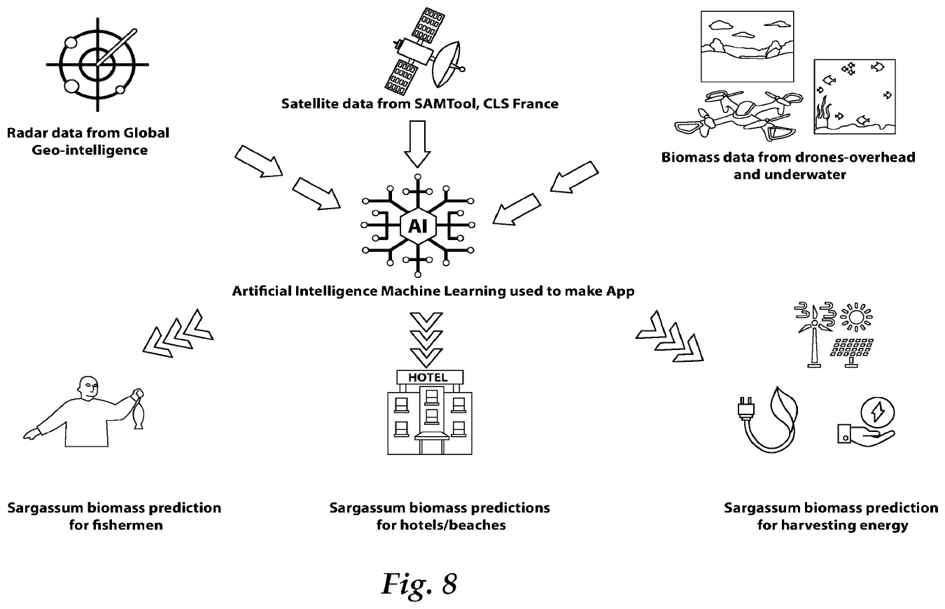

### DETAILED DESCRIPTION OF THE SEVERAL EMBODIMENTS

The detailed description set forth below in connection with the appended figures and tables is intended as a description of several embodiments of the composition and method of making same and does not represent the only forms in which the present composition and method may be constructed and/or utilized. The description sets forth the functions and the sequence of steps for the production of the composition in connection with the illustrated embodiments. However, it is to be understood that the same or equivalent functions and sequences may be accomplished by different embodiments that are also intended to be encompassed within the spirit and scope of the invention.

For the following defined terms, these definitions shall be applied, unless a different definition is given in the claims or elsewhere in this specification. All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

Reference will now be made to non-limiting embodiments, examples of which are illustrated in the Figures.

A biofuel composition and method of making same is disclosed herein, wherein the biofuel composition may be utilizing in numerous applications as an alternative fuel source, including but not limited to transportation vehicles. In one embodiment, the biofuel is composed from the co-digestion of three substrates, namely a quantity of rum distillery wastewater, a quantity of inoculum and a quantity of seaweed, preferably the genus sargassum.

In one embodiment, the process for producing the biofuel comprises placing a quantity of sargassum seaweed, a quantity of rum distillery wastewater and a quantity of inoculum into an anaerobic digester to breakdown the biodegradable materials to produce a quantity of biogas which may be utilized as a source of energy for an electrical grid among other things, or the biogas may be upgraded to biomethane by any standard commercial available system for hydrogen sulfide (H₂S), water and cardon dioxide (CO₂) removal. In turn, the biomethane may be compressed to utilize as alternative fuel in transportation vehicles.

The use of seaweed, in particular the genus sargassum has shown promise as use in generating biofuel, however the anaerobic digestion of sargassum seaweed into biomethane which may be used in transportation vehicles requires an additional element, namely water. It is known in the art that anaerobic digestion normally requires up to 7% solid feedstocks, which the remaining 93% of the digestate is essentially water (Parkin et al. 1986). Therefore, while the sargassum seaweed may provide all of the needed solid matter to aid in the production of the biofuel, a source of water would still be required which may be met by industrial wastewater, in particular water that is produced daily at local rum distilleries. In general, rum distillery wastewater is effectively high in chemical oxygen demand (COD) (Tauseef et al. 2013), which enhances the process of anaerobic digestion.

Sargassum is a genus of large brown seaweed that is abundant in the ocean and floats in masses and never attaches to the seafloor making it suitable for harvesting and use in the biofuel composition of the present disclosure. In identifying and sustainably harvesting fresh floating sargassum seaweed for use in the biofuel composition, an electronic prediction application, powered by a machine learning model trained on historical satellite data, radar data and near-field drone captures of arriving Sargassum mats and predicts the biomass amount, content and weight. Preferably, in collecting the quantity of sargassum, one would target primarily the problematic Sargassum seaweed mats, based on the biomass content prediction from the mobile application, with a predicted drift path that arrives within four to six days. Moreover, it would be preferable to select a location for collection that is accessible within a six-hour freight to the location of a biogas plant that will be utilized for the co-digestion and subsequent production of biogas and biofuel.

In a preferred embodiment, the quantity of sargassum seaweed is selected from the pelagic sargassum species, in particular, including, but not limited to the species Sargassum fluitans III, S. natans I, and S. natans VIII. Furthermore, in this embodiment, the harvested sargassum may be washed using ocean water or fresh water prior to transport to a biogas plant for introduction into the anaerobic digester. Ideally the harvested and washed sargassum is transported and placed in the digester within six hours following collection, however alternatively, in the event this is not feasible, the harvested sargassum may be stored at a temperature of about 25° C.

Separately, the quantity of rum distillery wastewater includes several components depending on the source of the distillery. In one embodiment, the rum distillery wastewater comprises a solution with the following ranges and elements:
(A) Ethanol: 1-10% (v/v)
(B) Organic acids (like aconitic acid, acetic acid, etc): less than 5% (v/v)
(C) Sugars (like sucrose and glucose, etc): less than 5% (w/w)
(D) Nitrogen compounds (like ammonia, nitrates etc): less than 1% (w/w)
(E) Salts and minerals (Calcium carbonate, Potassium chloride, Sodium chloride, etc): less than 1% (w/w)
(F) Heavy metals: Trace amounts to a few parts per million (ppm) or less
(G) H2O or water: at least 80% (v/v)
(H) Total microbial content in the wastewater above 10\hat{ }6 colony-forming units (CFUs) per milliliter (mL), including but not limited to Lactobacillus, and Bacillus species, yeast species such as Saccharomyces cerevisiae, Candida tropicalis, and/or Zygosaccharomyces bailii, and fungi.
(I) Biochemical oxygen demand (BOD) range above 30,000 mg/L
(J) Chemical oxygen demand (COD) range above 70,000 mg/L
(K) Phosphorous 0.005% or less
(L) Potassium-0.9% or less
(M) Calcium-0.2% or less

Lastly, the third component of the composition to generate biofuel is an inoculum, which generally is classified as a population of microorganisms or cells that is introduced in a fermentation medium. In one embodiment, the quantity of inoculum is primarily a biological bacteria source which may include either individually, or in combination, one of lionfish, Barbados sour grass, grass-fed blackbelly sheep manure and/or any other similar grass-fed ruminant manure.

In combination, the composition in one embodiment includes the quantity of rum distillery wastewater in the range of 80-98% w/w; the quantity of inoculum is in the range of 0.5-5% w/w and the quantity of sargassum seaweed in the range of 1-5% w/w. In yet another embodiment, the composition includes the quantity of rum distillery wastewater in the range of 90-96% w/w; the quantity of inoculum in the range of 2-4% w/w and the quantity of sargassum seaweed in the range of 2-4% w/w. In another embodiment, the composition includes the quantity of rum distillery wastewater of about 95% w/w; the quantity of inoculum of about 1.5% w/w and the quantity of sargassum seaweed of about 3.5% w/w.

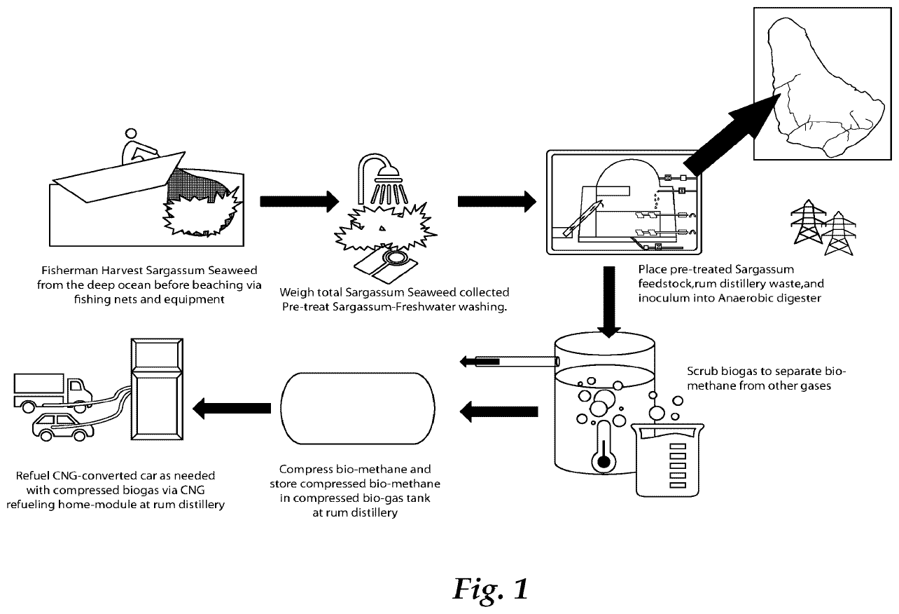

FIG. 1 illustrates an exemplary embodiment of the overall methodology and process for the production of biofuel according to the present disclosure, wherein as discussed above, the sargassum is located, harvested and washed prior to transport for inclusion in an anaerobic digester in combination with the inoculum and sargassum for the creation of biogas, and optionally to compress the biogas into biomethane for use as an alternative fuel source.

### EXAMPLES

Practical and presently preferred embodiments of the present disclosure are illustrated as shown in the following Examples. However, it will be appreciated that those skilled in the art, on consideration of this disclosure, may make modifications and improvements within the scope of the disclosure.

Table 1 shows a non-limiting overview of various components that may be used in the presented disclosure.

TABLE 1

| | Controls | | | Samples | | | | | |
|---|---|---|---|---|---|---|---|---|---|
| Components | 1 | 9 | 10 | 2 | 3 | 5 | 12 | 6 | 13 |
| Sargassum | yes | | | | | yes | yes | yes | yes |
| Saltwater pre-treatment | yes | | | | | yes | | yes | |
| Fresh water pre-treatment | | | | | | | yes | | yes |
| Low COD (Mount Gay) | | yes | | yes | | yes | yes | | |
| wastewater | | | | | | | | | |
| High COD (Foursquare) | | yes | yes | | yes | | | yes | yes |
| wastewater | | | | | | | | | |
| Bacteria source (Inoculum) | yes | | | yes | yes | yes | yes | yes | yes |

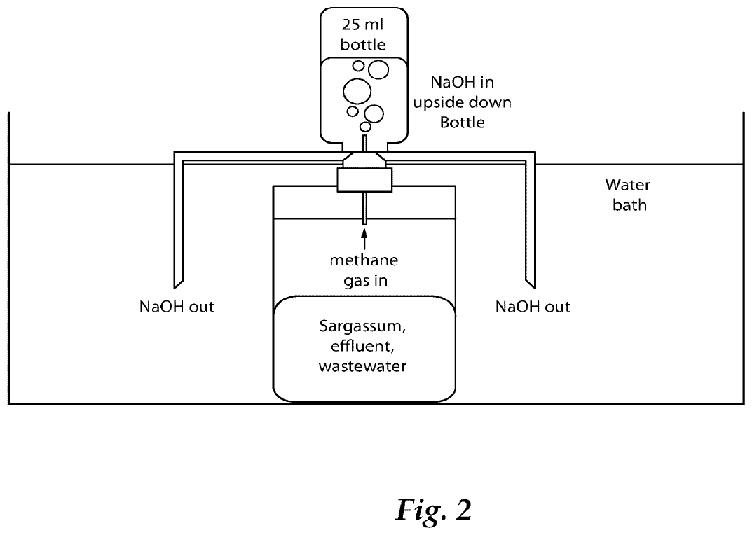

An exemplary experimental set-up for anaerobic digestion is shown in FIG. 2 for a biomethane potential test utilizing a micro-digester set-up taken from Holder et al. 2019. In the experimental set-up illustrated in FIG. 2, a small amount of rum distillery waste (25 ml) is placed into the sealed jar alongside 25 ml of inoculum (a mixture of Barbados sour grass and fish offal) and 3.5 g of fresh Sargassum seaweed undergoing various pre-treatments as described in Table 1. The different components of each treatment are presented on the left-hand side, while the right-hand side shows which components are applied to the controls and to the samples, respectively. The numbers on the first line of the right-hand side help refer to a specific control or sample. For instance, control 1 included Sargassum, saltwater pre-treatment and a bacteria source (inoculum).

In this setup, fresh sargassum seaweed is collected and washed with freshwater and stored at approximately 25° C. Forty empty jars are weighed prior to the addition of any samples. The fresh Sargassum samples are then added to the jars and the jars are weighed again to verify a mass of 3.5 g of fresh matter has been added to each jar. Then, the jarred samples are dried for 24 hours at 100° C. and then weighed again in the jar to record the weight of the matter when dry. The dried matter is then removed from the jars to be mechanically ground to 10 mm size particles. Once ground up, the matter is once again added back to the jars, along with a small amount of inoculum and 25 mL of distillery wastewater.

In this experimental set-up, the biomethane potential test is performed in the micro-digester set-up illustrated in FIG. 2, as described in (Holder et al. 2019). As shown in Table 1, all samples and controls went through this process before results could be observed.

The following describes the outcomes of the experiments executed for the different samples. The letter A, B, C and D are used to refer to the replicates of each sample in Table 1.

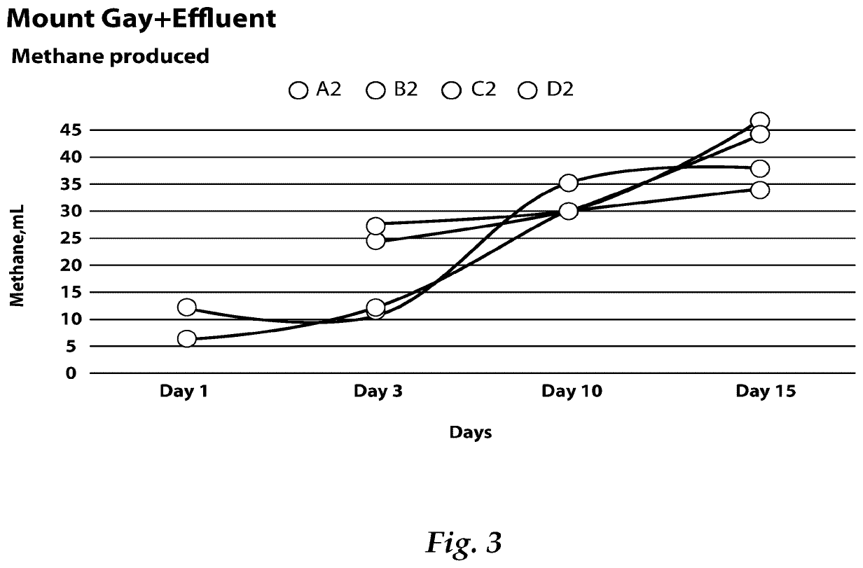

FIG. 3 shows the results of four replicates, A, B, C and D, of a biomethane potential test of Distillery low COD wastewater, and bacteria-rich inoculum with no Sargassum seaweed, which corresponds to sample 2 in Table 1. FIG. 3 further represents the volume of methane (Nml/gFM) produced from sample 2 of Table 1, where the x-axis represents the number of days and the y-axis represents the production of methane. As shown in FIG. 3, a fifteen-day rise reaches a methane production of 45 Nml/gFM by day 15.

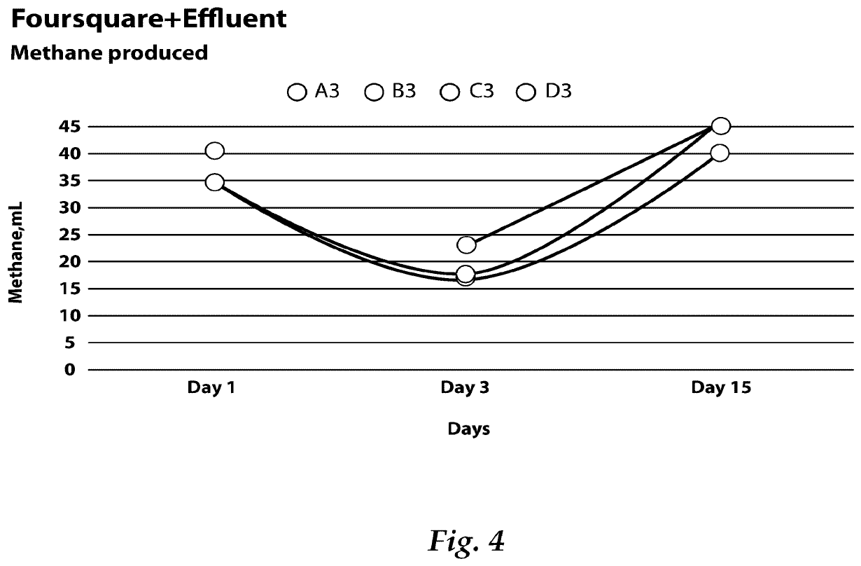

FIG. 4 shows the results of four replicates, A, B, C and D, of a biomethane potential test of Distillery high COD wastewater, and bacteria-rich inoculum with no Sargassum seaweed, which corresponds to sample 3 of Table 1. FIG. 4 further represents the volume of methane (Nml/gFM) produced from sample 3 of Table 1, where the x-axis represents the days and the y-axis represents the production of methane. As shown in FIG. 4, 45 Nml of methane was produced as early as day 1.

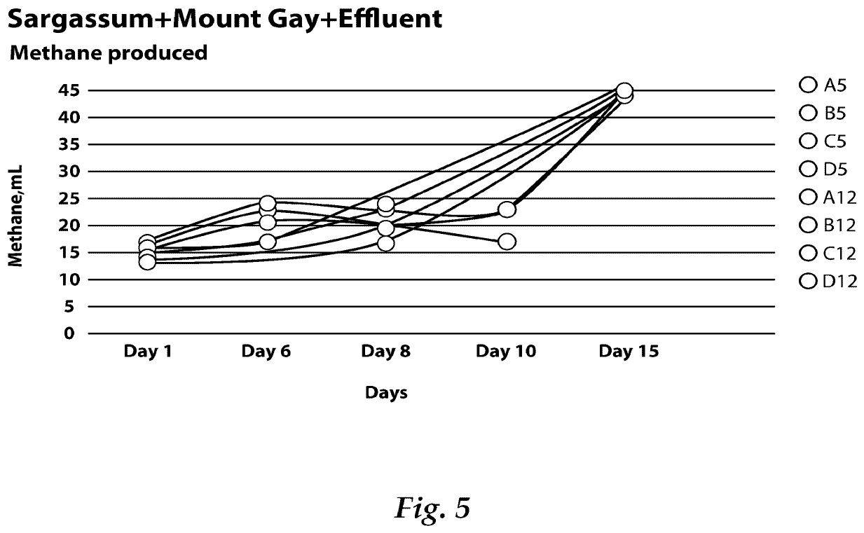

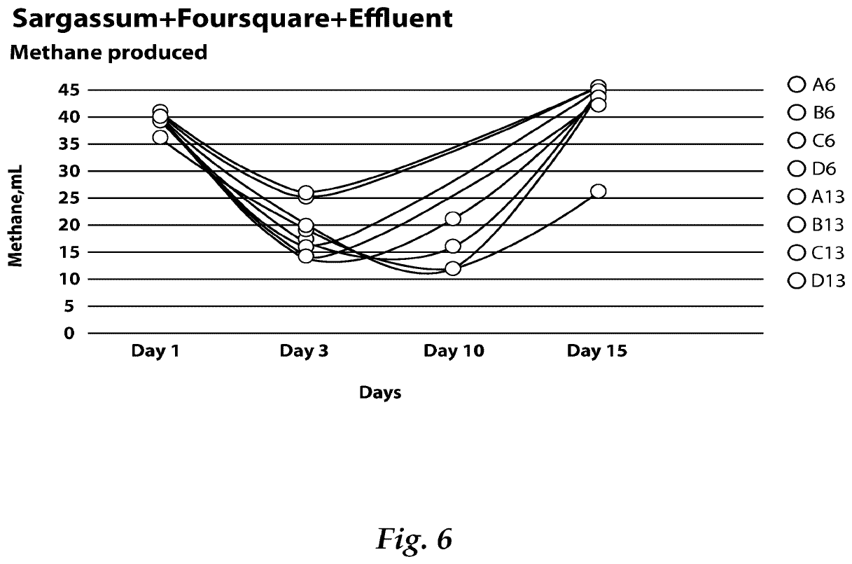

Comparing the results of FIG. 3 and FIG. 4, it can be observed that the level of COD in wastewater is demonstrated to influence the rate of methane production in the first 15 days of the experimental set-up. In the absence of Sargassum seaweed, it can be observed that the methane production of rum distillery waste is less predictable and more varied, as data lines across experimental replicates show greater spread in these images when compared to FIGS. 5 and 6.

FIG. 5 shows the results of four replicates, A, B, C and D, of a biomethane potential test of Distillery low COD wastewater, and bacteria-rich inoculum, which corresponds to samples 5 and 12. Samples 5 and 12 included pre-treated Sargassum seaweed, washed with salt-water and fresh water respectively.

FIG. 6 shows the results of four replicates, A, B, C and D, of a biomethane potential test of Distillery high COD wastewater, and bacteria-rich inoculum, which corresponds to samples 6 and 13. Samples 6 and 13 included pre-treated Sargassum seaweed, washed with salt-water and fresh water respectively.

Comparing the results of FIGS. 5 and 6, it can be observed that low COD wastewater and bacteria-rich inoculum with salt-water and fresh water pre-treated Sargassum seaweed produces methane in lower amounts than results from systems with high COD wastewater and bacteria-rich inoculum with salt-water and fresh water pre-treated Sargassum seaweed. It can be further observed in FIGS. 5 and 6 that behaviours are converging across replicates and even across different conditions, showing that Sargassum seaweed combined with rum distillery wastewater makes for predictable biomethane output.

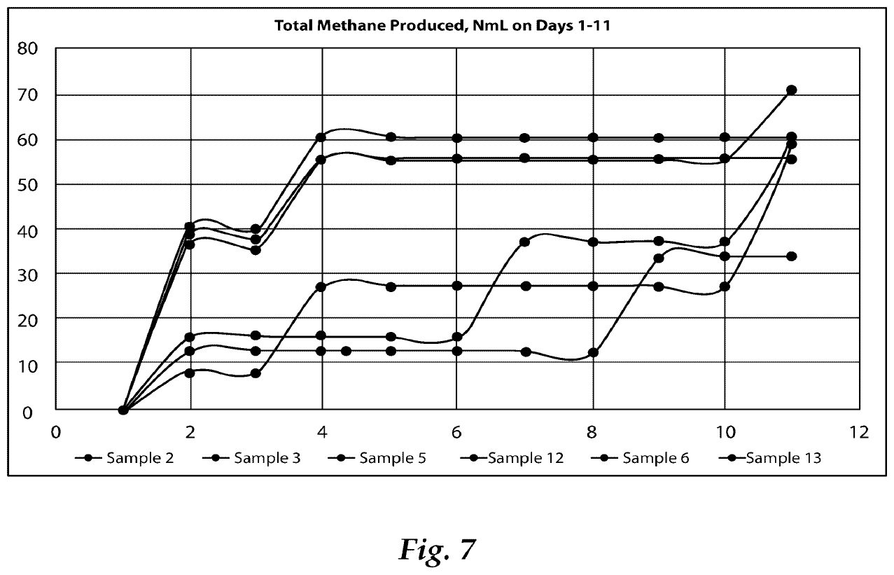

FIG. 7 shows the combination of all results from the different samples in Table 1 into a single figure. The best methane output comes from sample 13 of Table 1, with its high COD rum distillery waste combined with Sargassum seaweed that was pre-treated with freshwater. Overall, low COD levels in rum distillery wastewater coincide with less output biomethane with and without Sargassum seaweed.

Based on the results shown in FIG. 7, an approximate regression relationship between methane, M, and time, t could be generated, as follows:

$$M = {( 3.08 + 0.09 t )}^{3}$$

The results form the anaerobic digestion of Sargassum seaweed with rum distillery wastewater (Mckenzie et al. 2019) just presented above reveal the potential of Sargassum seaweed for rum-producing Caribbean nations.

### INDUSTRIAL APPLICABILITY

As explained hereinabove, biogas is produced by anaerobic digestion where micro-organisms breakdown organic matter (Dada et al. 2017). Once extracted, biogas can be scrubbed to remove carbon dioxide and any trace gases, such as hydrogen sulfide and small amounts of other elements. The biogas can then be upgraded, and the resulting product is biomethane, a gas consisting of 95% to 97% methane and 1% to 3% carbon dioxide (Ryckebosch et al. 2011)

Using the results from FIGS. 3, 4, 5, 6 and 7, it is possible to estimate the production of methane after 15 days of anaerobic digestion of high COD rum distillery wastewater and inoculum (a mixture of Barbadoes sour grass and fish offal). The outcome is an average of 43.75 Nml·gFM=4.375×10-5 Nm³ of methane gas produced by 25 ml of wastewater in a micro-digester. From the equation M=(3.08+0.09t)³, it can be estimated that approximately 185 Nml of biomethane can be produced in 30 days from this set up. Using the ideal gas law, that amounts to 0.00815 moles of biomethane gas.

What is claimed is:

1. A composition for anaerobic digestion for the production of biofuel comprising:
a quantity of sargassum seaweed;
a quantity of rum distillery wastewater; and
an inoculum.

2. The composition of claim 1, wherein the sargassum seaweed is a species selected from the group consisting of: Sargassum fluitans III, S. natans I and S. natans VIII.

3. The composition of claim 1, wherein the inoculum is selected is from one or a combination of lionfish, Barbados sour grass, blackbelly sheep manure or any other ruminant manure.

4. The composition of claim 1, wherein the rum distillery wastewater further comprises:
a quantity of ethanol;
a quantity of organic acids;
a quantity of sugars;
a quantity of nitrogen compounds;
a quantity of salts and minerals; and a
a quantity of water.

5. The composition of claim 1, wherein the quantity of rum distillery wastewater is in the range of 80-98% weight by weight (w/v).

6. The composition of claim 1, wherein the quantity of rum distillery wastewater is in the range of 90-96% w.

7. The composition of claim 1, wherein the quantity of rum distillery wastewater is about 95% weight by weight.

8. The composition of claim 1, wherein the quantity of inoculum is in the range of 0.5-5% weight by weight.

9. The composition of claim 1, wherein the quantity of inoculum is in the range of 2-4% weight by weight.

10. The composition of claim 1, wherein the quantity of inoculum is about 1.5% weight by weight.

11. The composition of claim 1, wherein the quantity of sargassum seaweed is in the range of 1-5% weight by weight.

12. The composition of claim 1, wherein the quantity of sargassum seaweed is in the range of 2-4% weight by weight.

13. The composition of claim 1, wherein the quantity of sargassum seaweed is about 3.5% weight by weight.

14. The composition of claim 1, wherein the quantity of rum wastewater distillery in the range of 80-98% w/v; the quantity of inoculum in the range of 0.5-5% w/v and the quantity of sargassum seaweed in the range of 1-5% w/v.

15. The composition of claim 1, wherein the quantity of rum wastewater distillery in the range of 90-96% w/v; the quantity of inoculum in the range of 2-4% w/v and the quantity of sargassum seaweed in the range of 2-4% w/v.

16. The composition of claim 1, wherein the quantity of rum wastewater distillery of about 95% w/v; the quantity of inoculum of about 1.5% w/v and the quantity of sargassum seaweed of about 3.5% w/v.

17. A method for the production of biofuel utilizing the composition of claim 1, comprising the steps of:
collecting the quantity of sargassum seaweed;
washing the quantity of sargassum seaweed;
combining the quantity of sargassum seaweed with the quantity of rum distillery wastewater and the inoculum in an anaerobic digestor; and
collecting a quantity of biogas from the anaerobic digestor.

18. The method for the production of the biofuel of claim 17, further comprising the step of:
upgrading the quantity of biogas to a quantity of biomethane by the removal of hydrogen sulfide (H₂S), water and cardon dioxide (CO₂).

19. The method for the production of biofuel of claim 17, further comprising the step of:
utilizing a biomass prediction mobile application to predict the arrival of the quantity of sargassum seaweed, wherein the mobile application incorporates an algorithm generated by machine learning trained on satellite data, radar data and drone data of arriving sargassum seaweed.
